(19) United States
(12) Patent Application Publication
(10) Pub. No.: US 2023/0132409 A1
(43) Pub. Date: May 4, 2023

(54) IN-LINE MEASUREMENT AND/OR DETECTION OF ANALYTES, CONTAMINANTS, AND PHYSICAL CHARACTERISTICS IN BODY FLUID MANAGEMENT SYSTEMS

(71) Applicant: BrainSpace, Inc., Camden (DE)

(72) Inventors: Caitlin D.C. Morse, Woodinville, WA (US); Stephen A. Morse, Woodinville, WA (US)

(21) Appl. No.: 17/980,163

(22) Filed: Nov. 3, 2022

Related U.S. Application Data
(60) Provisional application No. 63/275,232, filed on Nov. 3, 2021.
(60) Provisional application No. 63/315,910, filed on Mar. 2, 2022.

(51) Int. Cl. A61M 27/00

(52) U.S. Cl. CPC A61M 27/006; A61M 2205/3334; A61M 2205/3303; A61M 2205/3306; A61M 2205/50; A61M 2230/63; A61M 2202/0464

(57) ABSTRACT

Body fluid management systems and associated methods include in-line measurement and/or detection of analytes, contaminants, and/or physical characteristics for the diagnosis or detection of disease, infection, or toxicity.

### BACKGROUND

### Technical Field

The disclosure relates generally to body fluid management systems and associated methods.

### Description of the Related Art

Gravity-based drainage of bodily fluid has been practiced for centuries, starting with unrestricted drains, such as urinary catheters, intended to fully drain fluid from a body compartment. Controlled partial drainage of cerebrospinal fluid to relieve pressure on the brain has been used for almost 300 years and the art was further advanced in 1927 with the addition of fluid pressure measurement via a manometer. Manometers utilize the differential height of a column of fluid to measure pressure and require alignment to an anatomical marker. Despite these advances, common practice today includes the combination of a variety of devices including manometer-based drains, external pressure transducer assembles, arterial blood pressure modules attached to bedside patient monitors, and other equipment requiring complex and frequent management by the clinical user to manually switch between devices, extract samples, reset the system every time the patient moves or frequently recalibrate due to pressure sensor inaccuracy.

Current methods of analyzing body fluid suffer from slow and/or subjective processes that introduce unnecessary risk to patients. Physical analysis of body fluid is often performed as a bedside visual assessment with high interrater variability. Conclusions can vary significantly depending on the attentiveness, training, and eyesight of the observer and the conditions under which the observations are performed (lighting, background, ambient temperature, etc.). Chemical analysis of body fluid is typically done by withdrawing a sample from a patient and sending the sample to a lab. The current process is cumbersome, prone to error (sample mix-ups, contamination, etc.), and dangerously slow for critically ill patents.

### BRIEF SUMMARY

Disclosed herein are body fluid management systems and associated methods that, in addition to performing pressure monitoring, and body fluid drainage, also include sensing assemblies capable of in-line detection of analytes and physiological parameters of the target body fluid and motion sensing of the body. This may include the presence and/or concentration of bacteria, viruses, proteins, chemicals, elements, hormones, or other biomarkers or contaminants in a target body fluid; physiological parameters such as fluid opacity, particulate count, and temperature; or position and movement of the head, trunk, legs, feet, and more to determine body position, gait and movement intervals.

These sensing capabilities may offer qualitative or quantitative insight in real-time from the bedside and can be used in combination or separately to support local and remote collaboration with more rigor, precision and automation than is clinically available today. In certain embodiments, these sensing assemblies introduce fluid analysis without breaching of a sealed system for sample collection. Wearable sensing assemblies also allow for continuous sensing and trend analysis of parameters for which only intermittent sample analysis exists in the art. In certain combinations, they represent novel diagnostic medical systems capable of characterizing cerebrospinal fluid (CSF) volume and flow within the central nervous system and automating management of diagnostic procedures for medical conditions such as normal-pressure hydrocephalus, cerebrospinal fluid leaks, and related conditions of abnormal CSF circulation or volume.

Furthermore, many attempts are on-going to improve specific analysis capabilities, but a need remains in the art for a clinical deployment platform to expedite the commercialization path for novel sensing. The disclosed system introduces such a platform with a common body fluid management system with customizable and interchangeable sensor assemblies capable of communicating individual results separately or in combination to derive diagnostic results.

A body fluid management system may be summarized as comprising: a control system assembly for real-time monitoring of a pressure of a body fluid; a patient interface assembly comprising at least one wearable pressure sensor assembly; and at least one sensor assembly configured for detecting at least one parameter of the body fluid. The control system assembly may comprise integrated control of drainage of the body fluid. The wearable pressure sensor assembly may be configured for attaching proximate to a patient anatomical marker. The wearable pressure sensor assembly may comprise at least one pressure sensor in direct fluid communication with the body fluid. The control system may be configured for monitoring for changes in pressure of the body fluid. The at least one sensor assembly may be an analyte sensor assembly in direct fluid communication with the body fluid. The control system assembly may comprise integrated control of drainage of the body fluid. The analyte sensor may be disposed within a sealed fluid path of the body fluid. The analyte sensor assembly may be configured for detecting salinity of the body fluid. The analyte sensor assembly may be configured for detecting glucose level of the body fluid. The analyte sensor assembly may be configured for detecting protein concentration of the body fluid. The at least one sensor assembly may be a physiological sensor assembly. The physiological sensor assembly may be configured for detecting color of the body fluid. The physiological sensor assembly may be configured for detecting opacity of the body fluid. The physiological sensor assembly may be configured for detecting particulate in the body fluid. The physiological sensor assembly may be configured for detecting oxygen concentration in the body fluid. The control system may communicate a quantitative value derived from the sensor assembly. The control system may communicate a qualitative parameter from the sensor assembly.

A body fluid management system may be summarized as comprising: a control system assembly for real-time monitoring of a pressure of a body fluid and integrated control of drainage of the body fluid; a patient interface assembly comprising at least one wearable pressure sensor assembly; and at least one customizable sensor assembly configured for integration of an interchangeable sensor subassembly. The interchangeable sensor subassembly may be an interchangeable analyte detection subassembly. The interchangeable sensor subassembly may comprise a printed circuit board. The interchangeable sensor subassembly may communicate with the control system assembly.

A body fluid management system may be summarized as comprising: a control system assembly for real-time monitoring of a pressure of a body fluid and integrated control of drainage of the body fluid; a patient interface assembly comprising at least one wearable pressure sensor assembly; and at least one wearable motion sensor assembly. The patient interface assembly may comprise at least one orientation sensor configured to detect an orientation of a body cavity containing the body fluid and movement of the body cavity. The wearable motion sensor assembly may comprise at least one motion sensor configured to detect movement of the body. The at least one wearable motion sensor assembly may comprise two wearable motion sensor assemblies configured to be worn one on each ankle. The at least one wearable motion sensor assembly may comprise two wearable motion sensor assemblies configured to be worn one on each shoe. The at least one wearable motion sensor assembly may be configured to be worn at the lumbar region. The control system assembly may be configured to derive gait from patient interface assembly inputs. The control system assembly may be configured to detect normal pressure hydrocephalus that may be improved by a shunt.

### BRIEF DESCRIPTION OF THE SEVERAL VIEWS OF THE DRAWINGS

FIG. 1A illustrates a body fluid management system.

FIG. 1B illustrates another body fluid management system.

FIG. 1C illustrates another body fluid management system.

FIG. 1D illustrates another body fluid management system.

FIG. 2 illustrates a patient interface assembly of a body fluid management system.

FIG. 3 illustrates a top view of a wearable sensor assembly of a patient interface assembly of a body fluid management system.

FIG. 4 illustrates a side cutaway view of the wearable sensor assembly of FIG. 3.

FIG. 5A illustrates a fluid line of a body fluid management system.

FIG. 5B illustrates another fluid line of a body fluid management system.

FIG. 5C illustrates another fluid line of a body fluid management system.

FIG. 6 illustrates a body fluid management system with more than one fluid.

FIG. 7 illustrates a simplified operational diagram of a body fluid management system utilizing more than one body fluid and associated derived parameters to control drainage.

FIG. 8 illustrates a control system assembly of a body fluid management system.

FIG. 9 illustrates another view of the control system assembly of FIG. 8.

FIG. 10 illustrates a body fluid management system with an interchangeable sensor assembly.

FIG. 11 illustrates another body fluid management system with an interchangeable sensor assembly.

FIG. 12A illustrates another body fluid management system.

FIG. 12B illustrates a body fluid management system with assay interface assembly.

FIG. 13 illustrates an assay interface assembly.

FIG. 14 illustrates a simplified operational diagram of a body fluid management system utilizing sensing assemblies and associated derived parameters to control drainage.

FIG. 15 illustrates a body fluid management system with wearable motion sensor assemblies.

FIG. 16 illustrates a body fluid management system with more than one access location to the same fluid type.

FIG. 17A illustrates a first schematic of a wearable sensor assembly of a patient interface assembly of a body fluid management system.

FIG. 17B illustrates a second schematic of the wearable sensor assembly of FIG. 17A.

### DETAILED DESCRIPTION

The disclosed systems improve upon the existing art by facilitating consistent, rapid detection of target analyte(s) and/or contaminant(s) via a body fluid management and diagnostic platform that may include continuous pressure monitoring, controlled body fluid drainage, physiological sensing, motion sensing and customizable sensor assemblies with a common control and communication interface.

Disclosed are body fluid management systems and associated methods that incorporate in-line detection of analytes and/or contaminants in a target body fluid.

A body fluid in this context may include cerebrospinal fluid (CSF), blood, urine, wound exudate, interstitial fluid, and the like. Analytes in this context may include bacteria (or bacterial bi-products), viruses, cells (such as cancerous cells, inflammatory cells, leukocytes, white blood cells, or red blood cells), proteins (such as albumin, tau, hemoglobin, immunoglobin or other proteins associated with the presence of cancerous cells, infection or congenital disease), amyloid-beta peptides, prions, chemical compounds or molecules, chemical elements (such as heavy metals), hormones, biomarkers, glucose, lactate, pyruvate, gamma-aminobutyric acid (GABA), glutamate, sodium, potassium, creatine kinase, neurotransmitters, other body fluids (such as blood contaminating CSF or urine), exogenous compounds (such as pharmaceuticals) and the like.

The disclosed systems may utilize established analyte sensors with known clinical significance (such as electrochemical glucose sensors, conductivity or salinity sensors, urinary calcium sensors, chloride sensors, and the like) in novel ways. The disclosed systems may integrate emerging modalities such as lab-on-a-chip-based sensing of unprocessed whole blood or interstitial fluid.

Additionally, the disclosed systems may enable clinical evaluation of novel third-party sensors via the disclosed sensing platform including customizable sensor assemblies configured for operable communication with a common control system assembly.

Physiological parameters in this context may include color, opacity, turbidity, volume, flowrate, particulate count, and temperature. While body fluid pressure is a physiological parameter, in the context of this disclosure, it is called out separately. Pulse Pressure Variability, Brain Compliance and other derived information from the pressure waveform or changes associated with drainage may be considered physiological.

Wearable motion sensing assemblies, also known as patient movement tracking assemblies, motion tracking assemblies, motion sensor assemblies, kinesthetic sensing assemblies, etc. in this context are wearable assemblies measuring the orientation, position, and/or movement of a body, body cavity, body compartment, limb or other anatomical feature intended to be used in conjunction with a fluid management system to provide additional data sources for more complex analysis. These assemblies may include one or more orientation sensors, motion sensors, accelerometers, gyroscopes, contact force sensors, and the like. The use of the words motion or movement in this context may also refer to lack of motion, or the orientation or position of the body or its parts.

The control system assembly may communicate with sensor assemblies via common electrical interface(s) to obtain data from the sensor(s), and may present relevant information and/or recommendations to the user or third-party physical or virtual display or data repository (such as an EMR or database).

Customizable sensor assemblies may be configured to accept a variety of sensing elements, which may detect the presence and/or concentration of a variety of analytes or contaminants in a body fluid. In-line analyte sensor data may also be paired with physiological sensor data such as temperature, gyroscope, optical/visual, etc. and motion data including use of multi-axis accelerometer, as well as physical attributes of the patient (height, weight, mobility, etc.) for more complex considerations.

The control system assembly may utilize machine-learning (ML) algorithm(s) and/or image processing and/or computer vision and/or input(s) from other source(s), including user inputs and inputs from various sensing modalities included within the control system assembly (optical sensors, flow sensors, drip sensors, color sensors, cameras, temperature sensors, etc.), in combination with data from sensors contained within a customizable sensor assembly, to generate relevant information and/or recommendations to the user.

Sensors within a sensor assembly may comprise a variety of technologies and sensing modalities, including chemical, electrochemical, optical, and the like. Certain embodiments of the disclosed system may include “active” sensors which produce an electrical signal (voltage, resistance, capacitance, digital, etc.) or an optical signal (fluorescence, infrared, etc.) which may be read by the control system assembly. Other embodiments may include “passive” indicator(s) such as a material that changes color in the presence of a particular analyte, chemical, etc. Such materials may be affixed onto or embedded into a suitable substrate (paper, tubing, fabric, plastic, silicon, metal, etc.) by printing, bonding, infiltration, co-extrusion, vapor deposition, etc., as appropriate for the specific material/substrate combination. In the case of passive indicators, the disclosed system may include secondary sensors and circuitry to detect the output (such as a color change) of an indicator, or cameras to visually assess the change or the indicator may simply be in a location that is visually observable by the user.

In certain embodiments, the disclosed system may provide multimodal monitoring of analytes and/or derived parameters, such as the lactate pyruvate ratio, blood or tissue oxygenation, and the like. It may also compare analyte concentrations between two fluids to characterize their distribution within the body such as exogenous compound presence in blood relative to CSF to characterize the permeability of the blood-brain barrier, which is particularly relevant for pharmaceutical research, or the blood glucose/cerebral glucose ratio to determine patient-specific cerebral glucose levels or the albumin quotient comparing albumin levels in the blood and the CSF to assess blood brain barrier disruption. These sensors may be continuous and provide a trend or averaged value or they may be intermittent or single point in time. These values may be communicated to the user directly, used by the disclosed system as part of an alarm condition, or integrated with other inputs for screening, diagnosis, or prognostication. For example, abnormally low CSF glucose levels relative to a patient's blood glucose level is clinically significant and often associated with infection among other conditions. Therefore, a low CSF glucose value may be communicated via the graphical user interface or to a third-party monitor or database with or without alarms. In other embodiments, the disclosed system may evaluate measured values against a set threshold or trend curve to determine and communicate “suspected bacterial meningitis.”

In other embodiments, the disclosed system may provide diagnostic detection of the presence of antibodies, proteins, or similar biomarkers associated with diseases such as hospital acquired infections (C-diff, MRSA, etc.), leptomeningeal or other cancers, Alzheimer's disease, Parkinson's disease, or other neurodegenerative condition, or one or more of over 700 rare brain diseases. The disclosed system sensing assemblies may also be combined with other data sources to provide prognostication of these and other diseases such as limb-onset ALS where higher protein levels in the CSF are believed to indicate blood brain barrier disruption and are associated with higher fatalities.

Certain embodiments of the disclosed system may comprise a patient interface assembly and a control system assembly. In an embodiment of the disclosed system, the patient interface assembly is disposable (usable for a single patient) and the control system assembly is durable (usable for multiple patients). Sensors may be located within the patient interface assembly, located within the control system assembly, or provided as a separate assembly that may be durable or disposable depending on the type and construction of the specific sensor.

In other embodiments, the system may be fully disposable and communicate monitoring data and alarms to a third-party graphical user interface or database via wired or wireless connection.

In certain embodiments, the disclosed system may be configured to provide therapeutic drainage (i.e., removal of a body fluid from the body). In such embodiments, the control system assembly may comprise, in operable communication, a user interface, a primary flowrate control actuator, a secondary flow shutoff actuator, and a body fluid flow sensor (such as a drip detector, mass flow sensor, etc.).

In other embodiments, the disclosed system may be configured to manage more than one body fluid. These may be multiple fluids of a single patient or may include concurrent management of a single fluid in multiple patients. In such embodiments, the patient interface assembly may accommodate multiple patient tubing lines or the control system assembly may accommodate multiple patient interface assemblies. In such embodiments, one or more body fluids may be in fluid communication with sensors for monitoring purposes while one or more fluids may be monitored and therapeutically drained. Sensing of analytes in the fluid or physiological characteristics of the fluid may occur in one or more fluids.

In other embodiments, the disclosed system may be configured to recirculate a body fluid back into the body. In such embodiments, the control system assembly may additionally comprise a pump mechanism, heating and/or cooling elements, filtration element(s), and/or additional sensor(s), and the patient interface assembly may include additional fluid line(s) for patient connection at one or more access location(s).

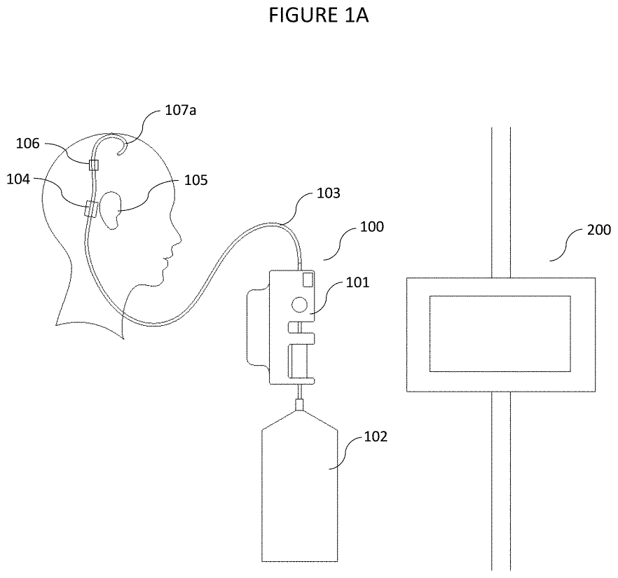

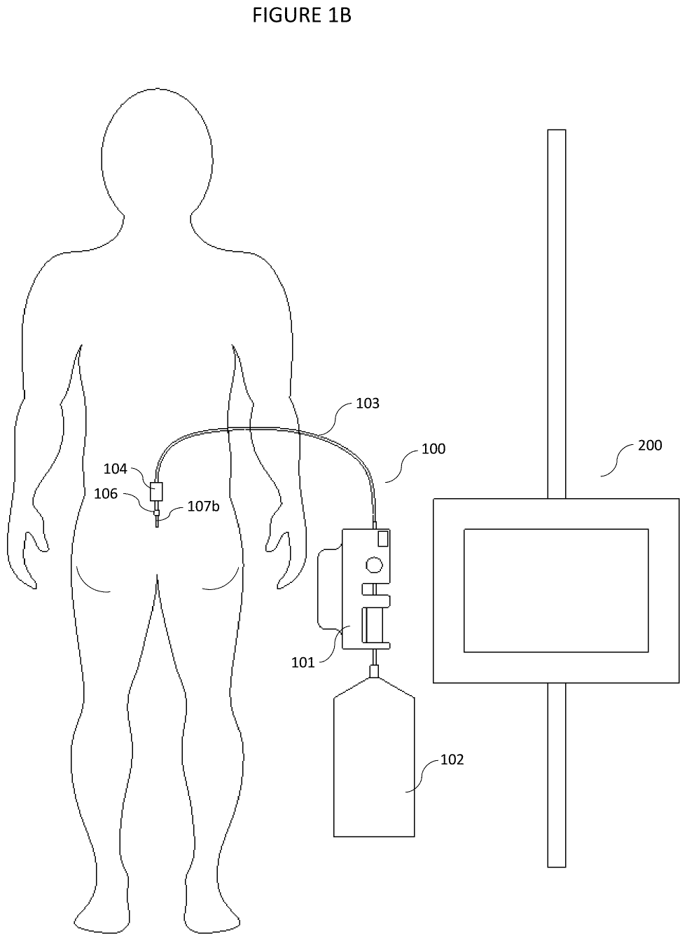

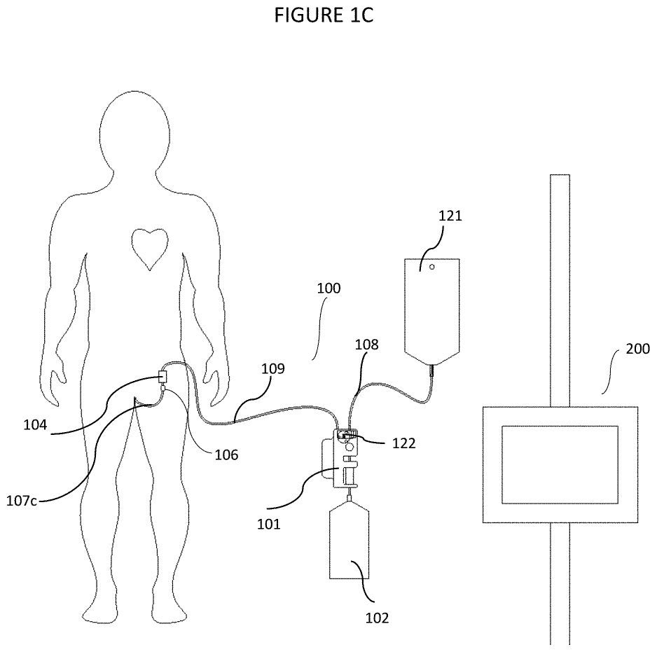

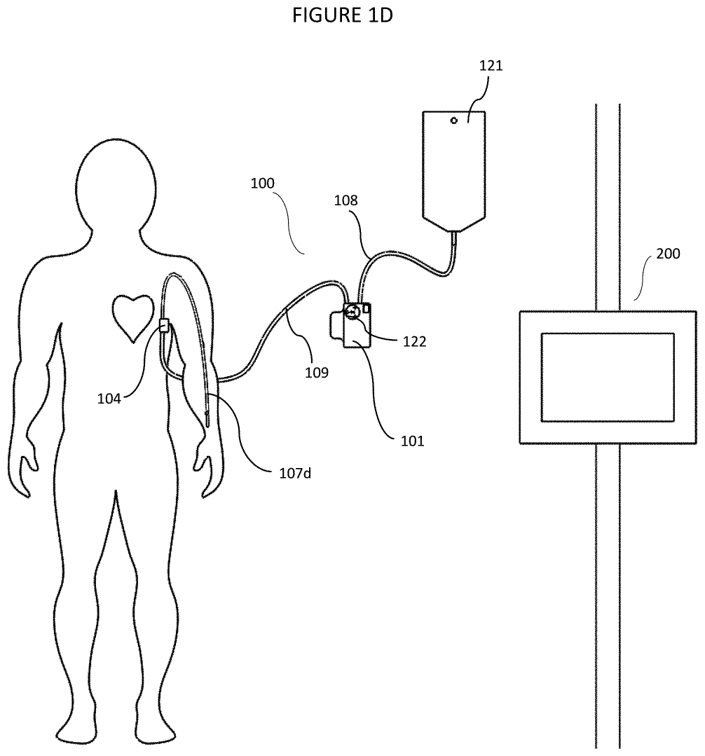

FIGS. 1A-1D are line drawings showing simplified diagrams of possible configurations of the disclosed system as it relates to patient anatomy, wherein the system is configured for monitoring and/or therapeutic drainage of body fluid and in-line detection of analytes in the body fluid, and wherein sensor assemblies are integrated within the patient interface assembly. FIG. 1A depicts an embodiment of the disclosed system that is configured for connection to a ventricular catheter for the monitoring, draining and analyzing of CSF. FIG. 1B depicts an embodiment of the system that is configured for connection to a lumbar catheter for the monitoring, draining, and analyzing of CSF. FIG. 1C depicts an embodiment of the system that is configured for connection to a urinary catheter for the monitoring, draining, and analyzing of urine. FIG. 1D depicts an embodiment of the system that is configured for connection to an invasive blood pressure catheter for the monitoring and analyzing of blood.

As depicted in FIG. 1A, the disclosed system comprises control system assembly 200 and patient interface assembly 100. Patient interface assembly 100 may comprise cartridge subassembly 101 for interfacing with the control system assembly 200, non-compliant body fluid drainage tube 103 and body fluid collection reservoir 102 for therapeutic drainage and collection of CSF, wearable pressure sensor assembly 104 configured for attachment to the patient proximate to a suitable anatomical marker 105 such as the external auditory meatus (EAM), and fluid-tight connector 106 (luer fitting, neuro fitting, etc.) for connection to implanted ventricular catheter 107a.

In related embodiments, the patient interface assembly may connect to a catheter implanted in the lumbar region of the spine (e.g., lumbar catheter 107b as depicted in FIG. 1B). In related embodiments, patient interface assembly may include wearable sensor assemblies worn at suitable anatomical markers for the pressure of the fluid being measured. When body fluid is blood, the intersection of the fourth intercostal space and the midaxillary line may serve as the anatomical marker of the heart. In other embodiments, the wearable pressure sensor assembly 104 may contain an orientation sensor, motion sensor, patient movement tracking assemblies, contact force sensor, or other sensing assemblies.

As depicted in FIGS. 1C and 1D, some embodiments may be configured for the use of an exogenous fluid 121 (saline, liquid pharmaceuticals, etc.) delivered through infusion line 108 to bidirectional non-compliant body fluid line 109 and may connect to a urinary catheter (e.g., a Foley catheter) 107c as depicted in FIG. 1C, an arterial catheter 107d as depicted in FIG. 1D, a venous catheter, or other similar device suitable for the target body fluid. Further embodiments may include the use of peristaltic pump 122 and/or a y-fitting within cartridge 101 to control infusion of exogenous fluid 121. In such embodiments as depicted in FIG. 1D in which the system is not configured for therapeutic drainage, body fluid-communicating sensor assemblies may be located in wearable pressure sensor assembly 104 or along bidirectional line 109.

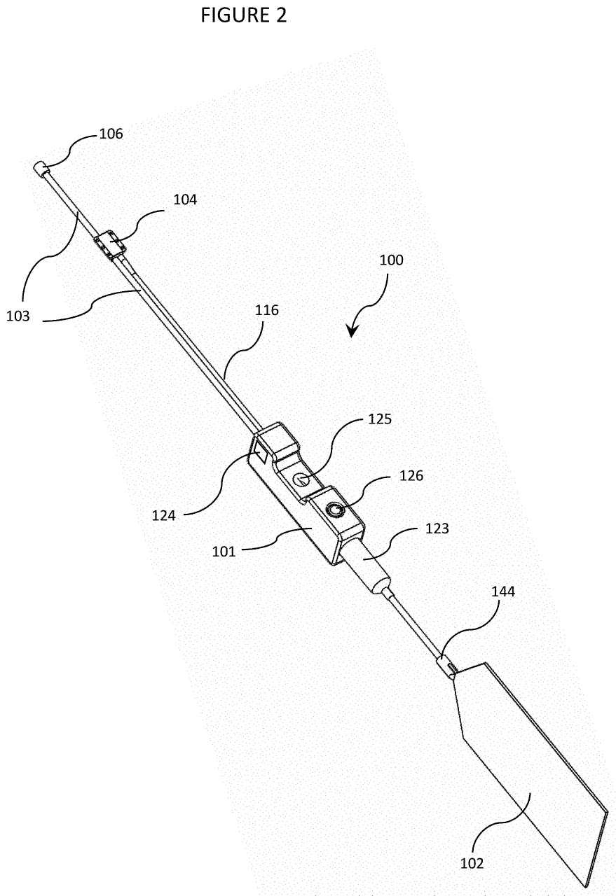

FIG. 2 is a line drawing showing a perspective view of patient interface assembly 100. As depicted in FIG. 2, patient interface assembly 100 may comprise flow measurement interface 123 (drip chamber, cuvette, tube, etc.) for interfacing with body fluid flow sensor 228 of control assembly 200, flowrate control actuator interface 125 (soft flexible tube, such as silicone, polyurethane, polypropylene-based elastomer, etc.) for interfacing with primary flowrate control actuator 222 within control system assembly 200, and flow shutoff actuator interface 126 (spring-loaded button, stopcock, pinch valve, etc.) for interfacing with secondary flow shutoff actuator 230 of control assembly 200. Patient interface assembly 100 may comprise drain tube 103 (silicone, polyurethane, polypropylene-based elastomer, etc.), configured at its inlet end with fitting 106 (luer fitting, neuro fitting, or other detachable fitting) for interfacing with an implanted catheter, and configured at its outlet end with fitting 144 for interfacing with detachable body fluid collection reservoir 102 (bag made of polyethylene, PVC, etc.) for collecting body fluids. Flow measurement interface 123 may comprise an orifice producing fluid droplets of a known size for accurate calculation of flowrate based on counting the number of fluid droplets detected by body fluid flow sensor 228 of control system assembly 200, and multiplying the number of droplets by the known droplet size.

Patient interface assembly 100 may also include electrical cable 116 between wearable pressure sensor assembly 104 and cartridge subassembly 101, and a set of exposed conductive pads 124 (gold, copper, carbon, silver ink, etc.) on cartridge subassembly 101 for passing electrical signals, data, power, etc. between patient interface assembly 100 and control system assembly 200. In such embodiments, a corresponding set of spring contacts (pogo pins, battery-style contacts, etc.) in control system assembly 200 may interface with the conductive pads in the patient interface assembly. Other embodiments of cartridge subassembly 101 may alternatively comprise a traditional electrical connector that is manually inserted into a corresponding receptacle in the control system assembly 200 by the user. Yet other embodiments may replace the physical electrical interface altogether by implementing wireless communication (Bluetooth, Wi-Fi, etc.) between patient interface assembly 100 and control system assembly 200, or between patient interface assembly 100 and a remote control system (cloud-based system, on-site or off-site server, smartphone or tablet-based application, etc.). In such arrangements, wearable pressure sensor assembly 104 may be powered with a battery or similar power source.

In certain embodiments, the cartridge subassembly 101 may include a peristaltic pump or similar mechanism, or interface with such mechanism in the control system assembly 200. In other embodiments, the cartridge subassembly 101 may include additional fluid line(s) for connection to an infusion source and/or secondary or tertiary patient fluid catheters. In yet other embodiments, the patient interface assembly 100 may comprise a simple volume-limited reservoir that incorporates one or more of the sensors described herein.

The patient interface assembly 100 may comprise one or more sensor assemblies, including a customizable sensor assembly, or sensor assemblies may be a separate assembly from the patient interface assembly 100.

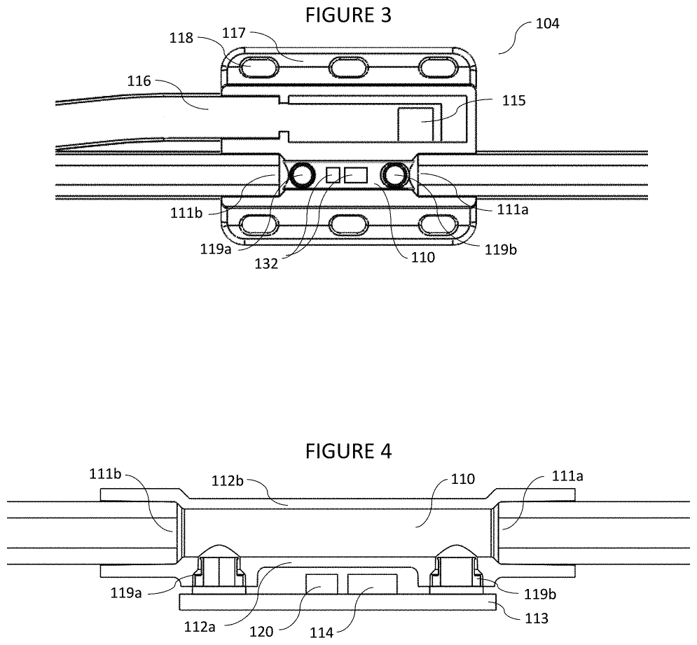

FIGS. 3 and 4 are line drawings showing top and side cutaway views, respectively, of embodiments of wearable pressure sensor assembly 104. FIG. 3 illustrates integration of fluid-communicating sensors in wearable pressure sensor assembly 104 and FIG. 4 illustrates integration of physiological, non-fluid-communicating sensors in wearable pressure sensor assembly 104.

As depicted in FIG. 3, wearable pressure sensor assembly 104 may comprise flow channel 110 with fluid inlet 111a and fluid outlet 111b opposite the fluid inlet 111a, pressure sensors 119a and 119b in fluid communication with the body fluid for measuring body fluid pressure, and orientation sensor 115 (e.g., accelerometer) for detecting patient movement. Wearable pressure sensor assembly 104 may further comprise housing 117 with suture points 118 for attaching wearable pressure sensor assembly 104 to the patient (as depicted in FIG. 1). Wearable sensor pressure assembly 104 may comprise one or more fluid communicating sensors 132 for the detection of analytes or physiological parameters such as temperature. Fluid communicating sensors may be electrical, such as for the measurement of salinity, or electrochemical, such as to detect glucose, or bioelectrical, such as a molecularly imprinted polymer membrane or other form of analyte sensor or biosensor known in the art.

As depicted in FIG. 4, certain embodiments of wearable pressure sensor assembly 104 may further comprise physiological sensor assemblies including an illumination source 120 (LED, etc.) and optical sensor 114 (such as color sensor AMS TCS34725 or similar, camera, etc.) for detecting changes in one or more physical attributes of the target body fluid (color, turbidity, etc.). The optical sensor 114 and illumination source 120 may be directed towards flow channel 110, which may comprise a transparent or semi-transparent side 112a facing the optical sensor 114 and illumination source 120, and an opposite side 112b providing a reflective surface or color reference (e.g., white) surface. In other embodiments, illumination source 120 and optical sensor 114 may be configured on opposing sides of flow channel 110. In related embodiments, optical sensor 114 may comprise an array of several optical sensors and illumination source 120 may comprise an array of several illumination sources. The illumination source(s) may be configured to illuminate the fluid in the flow channel at a particular wavelength, spectrum, or intensity of light conducive to detection of particular contaminant(s) by the optical sensor(s) (e.g., red or yellow tint indicating traces of blood or pus in normally clear CSF, dark yellow or brown tint indicating subpar kidney performance in otherwise pale-yellow urine, etc.). The illumination source(s) may be further configured to switch between various wavelengths, spectra, or intensities optimized for enhanced detection of certain contaminants sequentially or at the request of the control system assembly.

As depicted in FIGS. 3 and 4, the sensing assemblies including associated components (optical sensor 114, illumination source 120, orientation sensor 115, pressure sensors 119a and 119b, fluid communicating sensors 132, etc.) may be co-located on a printed circuit board (“PCB”) 113 (comprising a substrate of FR4, Kapton, polyester, etc.) and may utilize a single communication and power interface along electrical cable 116. In related embodiments, wireless technologies may be used to accomplish transmission of power (inductive or resonant-inductive coupling, etc.) and/or signal communication (RF, infrared, etc.). In other related embodiments, wearable pressure sensor assembly 104 may be powered internally (battery, capacitor, etc.). In further embodiments, more than one wearable sensor assembly may be located at any suitable location along body fluid drainage line 103 or bidirectional line 109.

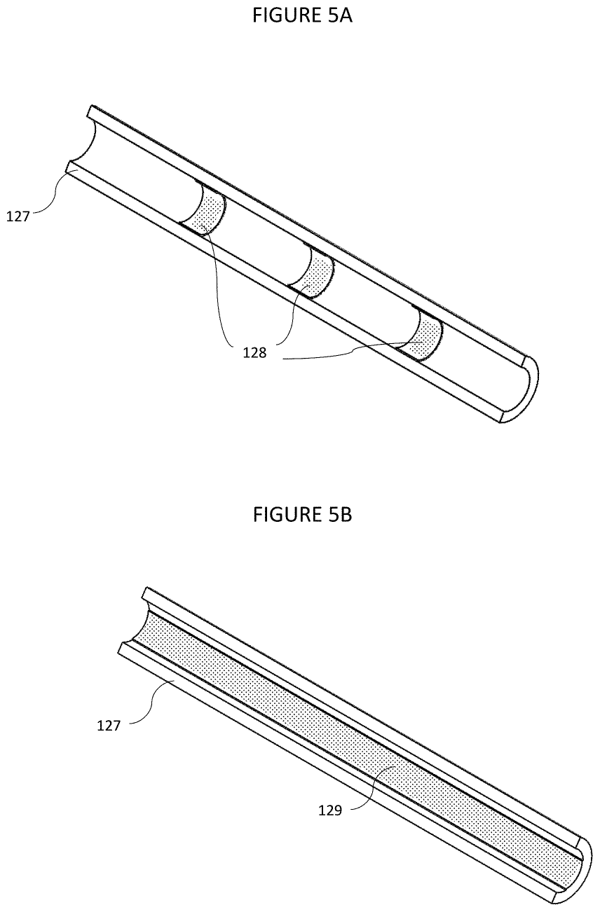

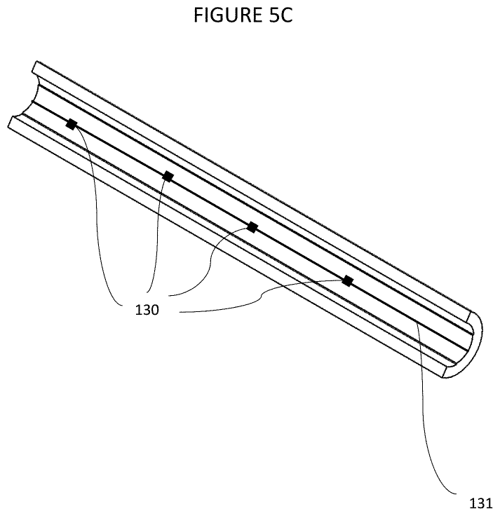

In other embodiments, as depicted in FIGS. 5A-5C, fluid-communicating sensor(s) optimized for the detection of one or more of the analytes or contaminants described elsewhere herein, may be located directly on and/or in fluid line 127 such as body fluid drainage line 103 or bidirectional line 109 at any suitable location. FIG. 5A depicts the use of sensing bands 128 such as colorimetric coating sensors delivering visually detectable results directly to the user. FIG. 5B depicts the use of a continuous sensing strip 129, which may comprise colorimetric coating as described herein or it may comprise a flex circuit adhered to the inner surface of fluid line 127 or suspended within the body fluid of fluid line 127.

In other embodiments, fluid line 127 fabrication itself may comprise a sensing material such as a leuco dye, which can serve as a colorimetric sensor due to its chemical form changing property caused by changes in heat, light or pH. In related embodiments, as depicted in FIG. 5C, electronic fluid-communicating sensors 130 may be embedded in the fluid line itself using overmolding, co-extrusion or other fabrication methods and an electrical cable 131 or flat flex circuit that may be fabricated within the tubing material and/or attached to the inside or outside of fluid line 127.

In other embodiments, sensors and/or illumination sources may be located at any other suitable position along the fluid line (e.g., within a second sensor assembly, within the cartridge, between the cartridge and the drainage reservoir, etc.). In yet other embodiments, sensors and/or their illumination source(s) that do not directly contact the fluid in the drainage line may be located within the control system assembly or provided as separate assemblies that may be used in operable communication with the control system assembly 200. In related embodiments, sensor(s) and/or illumination source(s) may be partially or fully enclosed in opaque or semi-opaque enclosure(s) to minimize and/or control interference from ambient light.

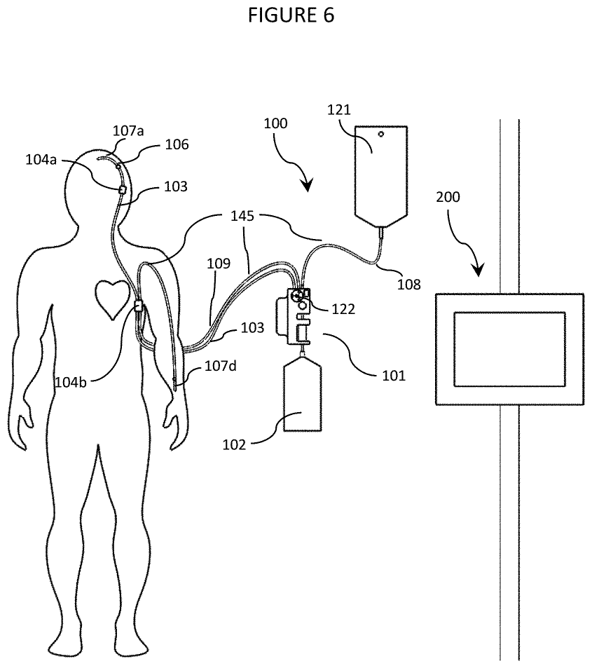

FIG. 6 is a line drawing depicting an embodiment of the presently disclosed body fluid management system wherein the patient interface assembly or assemblies are in fluid communication with more than one body fluid. As depicted in FIG. 6, patient interface assembly 100 may comprise cartridge subassembly 101 for interfacing with the control system assembly 200, non-compliant body fluid drainage tube 103 for connection to an implanted ventricular catheter 107a at its proximal end and a detachable fluid collection reservoir 102 at its distal end for therapeutic drainage and collection, wearable pressure sensor assembly 104a configured for attachment to the patient proximate to a suitable anatomical marker such as the external auditory meatus (EAM), and fluid-tight connector 106 (luer fitting, neuro fitting, stop-cock, etc.) for connection to implanted ventricular catheter 107a. Patient interface assembly 100 may further comprise a secondary fluid line 145 that may allow for the use of an exogenous fluid 121 (saline, liquid pharmaceuticals, etc.) delivered through infusion line 108 to bidirectional non-compliant body fluid line 109 and may connect at its proximal end to an arterial catheter 107d or other similar device suitable for the target body fluid such as a venous catheter, a urinary catheter, or the like. Further embodiments may include the use of peristaltic pump 122 and/or a y-fitting within cartridge 101 to control infusion of exogenous fluid 121. In embodiments in which secondary fluid line 145 is not configured for therapeutic drainage, body fluid-communicating sensor assemblies, such as analyte assemblies, may be located in wearable pressure sensor assembly 104 or along bidirectional line 109 as far as connector to arterial catheter 107d.

In other aspects, the patient interface assembly may further comprise first wearable pressure sensor assembly 104a, which is affixed to the patient substantially proximate to an anatomical marker appropriate for monitoring ICP (EAM), and second wearable pressure sensor assembly 104b, which is affixed to the patient substantially proximate to an anatomical marker appropriate for monitoring blood pressure (midaxillary line at the fourth left intercostal space, etc.).

Each of the first and second wearable pressure sensor assemblies may include an orientation sensor for monitoring patient movement/posture and error-checking pressure sensor readings as described elsewhere herein. The inclusion of multiple motion sensors or wearable motion sensing assemblies facilitates more detailed tracking of patient posture (e.g., tracking patient trunk orientation independent of head orientation for more accurate real-time modeling of the spinal column and associated CSF pressures in 3D space). Such information may be utilized by the system control assembly to automatically adjust displayed values to reflect the true value of a particular parameter more accurately at the anatomical point of interest, or for tracking of patient movement over time (e.g., for ensuring a patient is moved with sufficient frequency to prevent pressure injuries or for monitoring a patient that may be waking from a comatose or sedated condition). Additional physiological and analyte sensing assemblies may be located in wearable pressure sensor assemblies 104a and 104b, within or along drainage line 103 or 109, in cartridge 101 or within console 200.

In certain embodiments, the first wearable pressure sensor assembly 104a may monitor only ICP, whereas the second wearable pressure sensor assembly 104b may monitor both ICP and blood pressure. Such an arrangement provides a pressure reference for ICP that is normalized at the same elevation as the blood pressure reference for accurate calculation of cerebral perfusion pressure. In alternate embodiments, the second wearable pressure sensor assembly 104b may monitor blood pressure only. In certain embodiments, the disclosed system may use data from wearable pressure sensor assembly 104a and data from wearable pressure sensor assembly 104b and other sensing assemblies in combination to calculate a derived parameter. For example, the disclosed system may calculate real-time perfusion pressure, which is the net pressure gradient determining blood circulation through a given body compartment or body cavity. This may be calculated utilizing the mean arterial pressure measured by the wearable pressure sensor assembly 104b affixed to the patient substantially proximate to an anatomical marker appropriate for monitoring blood pressure and subtracting the body fluid pressure of the body cavity for which perfusion pressure is desired, also measured by 104b at the same blood pressure anatomical marker. Examples include cerebral perfusion pressure in which the body cavity is the cranial compartment, spinal cord perfusion pressure or lumbar perfusion pressure in which the body cavity is the spinal column, specifically the intrathecal space around the spinal cord, abdominal perfusion pressure in which the body cavity is the peritoneal cavity.

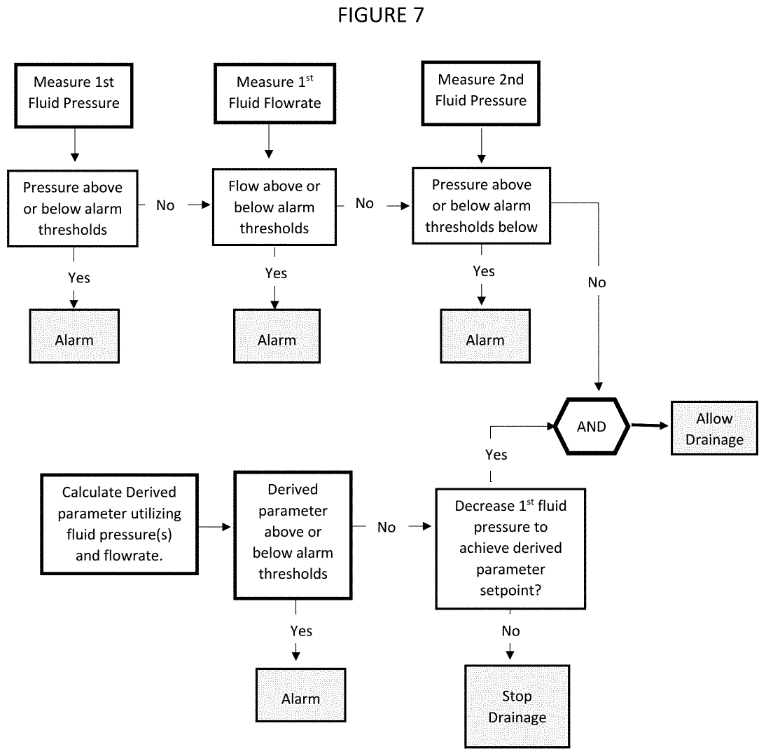

Further embodiments may monitor and/or control pressure or flowrate of a single fluid to achieve a target perfusion pressure rather than a target fluid pressure. The logic of one such embodiment is depicted in FIG. 7. Systems that automatically manage fluid pressure and drainage flowrate based on derived parameters (such as CPP) represent a significant advancement over the prior art, and enable exciting and useful clinical applications that are not possible or practical with existing technologies. Similarly, certain embodiments may control flowrate of a body fluid to achieve a target sensing parameter as described below.

In certain embodiments, the system may include a peristaltic or similar pumping mechanism for control of other fluids (saline, artificial CSF, etc.) known in the art for the purposes of periodic flushing, back-pressure (as may be the case with an arterial line), etc.

In certain embodiments, the patient interface assembly 100 may comprise multiple fluid lines as depicted in FIG. 6 or the control system assembly 200 may interface with multiple patient interface assemblies. These interfaces may be physical interaction such as a cartridge 101 insertion or they may be digital interactions such as wireless communication from sensing assemblies to control assembly or external display or database. In these multi-fluid embodiments, one or more body fluids may be in fluid communication with sensors for monitoring purposes while one or more fluids may be therapeutically drained in addition to monitoring and sensing. In other embodiments, the system may be configured to monitor or control two body fluids independently.

Sensing of analytes in the fluid or physiological characteristics of the fluid may occur in one or more fluids. Each parameter may be measured and reported individually. The disclosed system may include calculation of a derived parameter or compare analyte concentrations between two fluids to characterize their distribution within the body. Examples include detection of exogenous compound presence in blood relative to presence in CSF to characterize the permeability of the blood-brain barrier, or detection of blood glucose levels and cerebral glucose levels to calculate blood/cerebral glucose ratio to determine whether patient-specific cerebral glucose levels are in the expected range.

In some embodiments comprising multiple fluid lines, the patient interface assembly 100 may be comprised of a single integrated assembly, whereas in other embodiments the primary, secondary, and tertiary lines and their associated components may be separate patient interface assemblies. In some embodiments, various aspects of pumping mechanism 122 may be divided between patient interface assembly 100 and control system assembly 200.

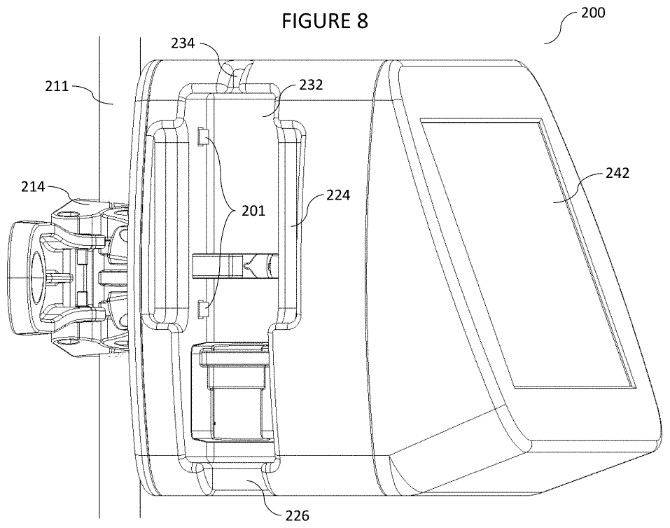

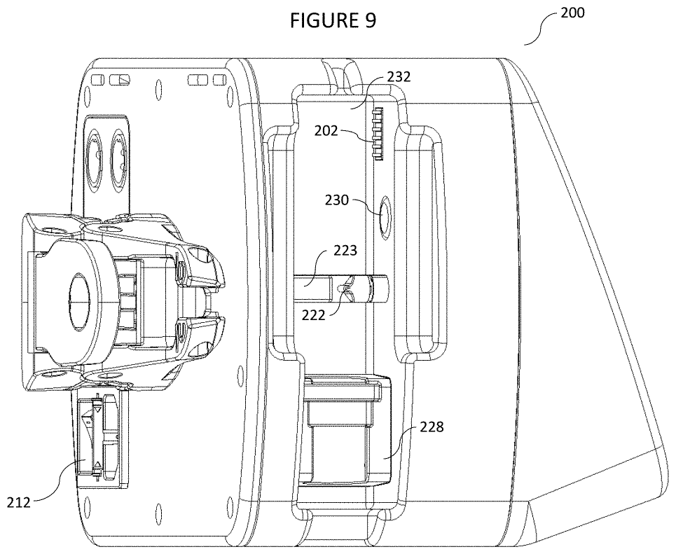

FIGS. 8 and 9 are line drawings showing perspective views of an embodiment of control system assembly 200 that is configured for use in operable combination with a patient interface assembly.

As depicted in FIG. 8, control system assembly 200 may comprise cartridge recess 232, fluid tube inlet recess 234, fluid tube outlet recess 226, and cartridge latching mechanism 201 for aligning and retaining the cartridge subassembly 101 within the control system assembly 200 during system operation. In certain embodiments, cartridge recess 232 may further comprise finger/hand clearance feature(s) 224 to facilitate insertion and removal of the cartridge subassembly 101 by the user.

Control system assembly 200 may further comprise user interface 242 for receiving user input (settings, patient information, etc.) and displaying system settings and outputs (set points, alarm thresholds, current or historical pressure or flowrate data, alarms, notifications, waveforms, patient information, etc.). In certain embodiments, user interface 242 may comprise a graphical display (LCD, OLED, etc.), a touchscreen (resistive, capacitive, projected capacitive, etc.), a button keypad (plastic or elastomeric buttons, membrane switch, etc.), an LED array (7-segment, individual indicators, etc.), or any similar elements suitable for entry of user inputs and display of system settings and outputs. In other embodiments, this user interface for receiving user inputs and displaying system settings and outputs may be located on a web browser, smartphone, external monitor, or other third-party device in communication with the control system assembly.

Control system assembly 200 may further comprise adjustable clamping mechanism 214 for fixation to an IV pole 211, bed rail, or other similar patient room furnishing. In other embodiments, control system assembly 200 may be configured to be cart-mounted, wall-mounted, free-standing, or secured in other ways within a transport vehicle including at an angle non-parallel to the ground to compensate for vehicle movement. In other embodiments, control system assembly may be configured to be carried with a handle or placed on patient bed or mobility device during transport.

As depicted in FIG. 9, control system assembly 200 may comprise receptacle 212 for connection to an external power source (AC mains, DC network, etc.), and may optionally include an internal power source (e.g., a rechargeable battery).

In further aspects, control system assembly 200 may comprise electrical interface 202 (connector/socket, pogo-pin array, spring-loaded contact array, etc.) for DC power distribution and electrical signal communication with the patient interface assembly. In other embodiments, such communication or power distribution may be accomplished wirelessly.

In other aspects depicted in FIG. 9, control system assembly 200 may include, in operable combination, primary flowrate control actuator 222 and backstop 223 for controlling flowrate of a body fluid, secondary flow shutoff actuator 230 for automatic shutoff of drainage flow in the case of power loss or system failure, and body fluid flow sensor 228 for detecting drainage flowrate.

In certain embodiments, body fluid flow sensor 228 may be an optical sensor for detecting falling fluid drops (as in drops falling through a drip chamber, cuvette, or similar enclosure), a mass flow sensor, an ultrasonic flow sensor, or any other similar sensor that is capable of detecting flow of the target body fluid with clinically-acceptable precision and accuracy.

In certain embodiments, secondary flow shutoff actuator 230 may be a DC motor with encoder and leadscrew, a stepper motor with leadscrew, a servo motor, a solenoid, a linear actuator, an electromagnetic latch, or any other similar actuator or latching mechanism that can be actuated sufficiently rapidly to shut off flow in the case of power loss or system failure.

In certain embodiments, primary flowrate control actuator 222 may be a DC motor with encoder and leadscrew, a stepper motor with leadscrew, a servo motor, a solenoid, a linear actuator, or any other similar actuator that either provides precise positioning for substantially constant flowrate (as in the case of a motor with encoder, stepper, or servo) or can be actuated rapidly between on/off states for intermittent flow (as in the case of a solenoid).

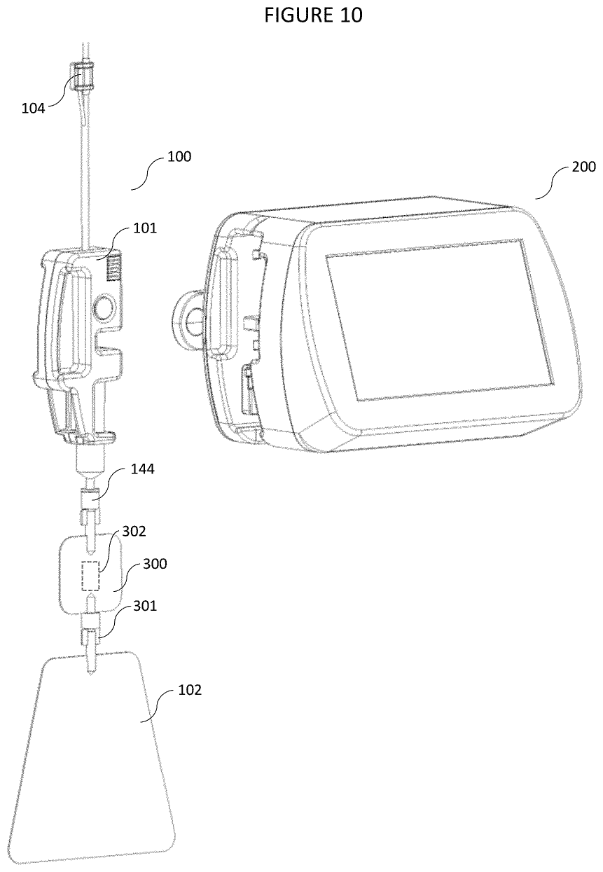

FIGS. 10-13 are line drawings showing embodiments of the disclosed system comprising customizable sensing assemblies wherein detection of one or more analytes is accomplished with the use of interchangeable subassemblies creating a sensing platform.

As shown in FIG. 10, interchangeable analyte detection subassembly 300 may be connected in-line between cartridge subassembly 101 and body fluid collection reservoir 102 by fluid-tight fittings 144 and 301. The interchangeable analyte detection subassembly may comprise indicator strip 302 that is coated or impregnated with a reagent formulated for a target analyte. In such embodiments, the indicator strip may remain a starting color (e.g., white) until the target analyte reacts with the reagent, which causes the indicator strip to change color (e.g., blue, red, etc.) without changing the color of the fluid in body fluid collection reservoir 102. In related embodiments, indicator strip 302 may comprise a solid plate enzyme-linked immunosorbent assay (ELISA) to detect a protein using an antibody response.

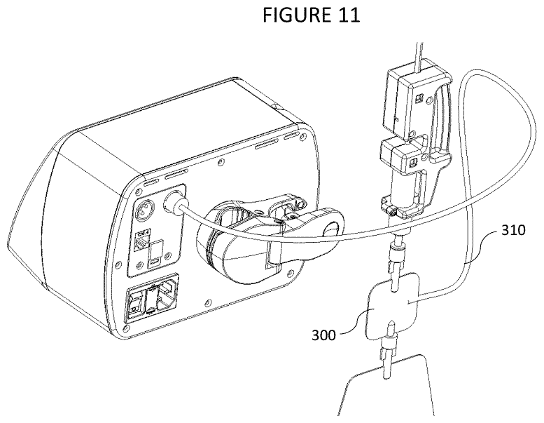

As shown in FIG. 11, interchangeable analyte detection subassembly 300 may comprise a lab-on-a-chip sensor or other electrical or electrochemical sensor mounted to a PCB or PCBA, powered by and communicate results to the control system assembly 200 via electrical cable 310. Alternatively, interchangeable analyte detection subassembly may communicate to the control system assembly or external display or database via wireless (Wi-Fi, Bluetooth, etc.) signal(s).

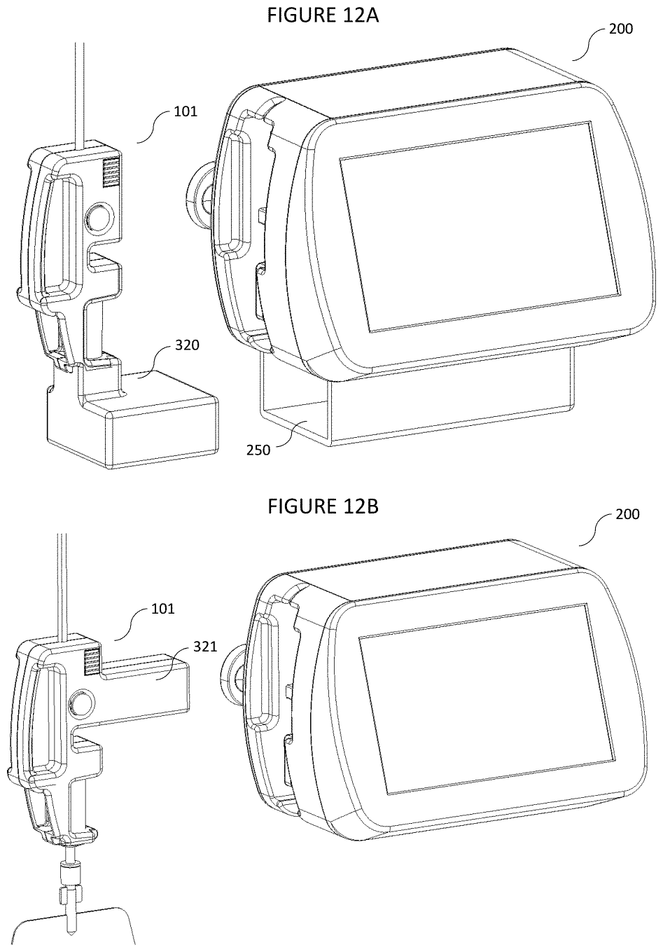

FIG. 12A is a line drawing showing an embodiment of the disclosed system wherein the drainage reservoir is enclosed within the system.

As depicted in FIG. 12A, cartridge subassembly 101 may comprise drainage reservoir enclosure feature 320 for a drainage reservoir (such as a drain bag) which slides into mating enclosure feature 250 within control system assembly 200. Drainage reservoir enclosure feature 320 may comprise a removable portion (not depicted) for accessing and/or replacing the drainage reservoir. In other embodiments, the drainage reservoir enclosure feature 250 may be an open-frame structure that retains the drainage reservoir 320 or body fluid collection reservoir 102 without fully enclosing it, such that the drainage reservoir and its connection(s) are readily visible and/or accessible to the user.

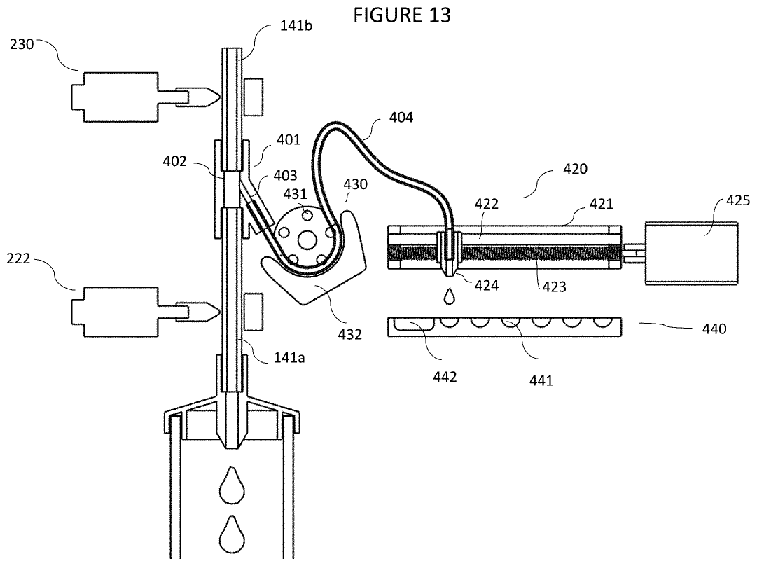

FIG. 12B is a line drawing showing an alternate embodiment of the disclosed customizable sensing assembly comprising a microfluidics assay interface. As depicted in FIG. 12B, cartridge subassembly 101 may comprise assay interface assembly 321 comprising a body fluid delivery mechanism and one or more sensing assemblies. Control system assembly 200 may also include access for compatible third-party microfluidics chamber and lateral flow assay, direct or indirect ELISA assay, or other biosensors to be separately inserted. FIG. 13 is a line drawing depicting one such embodiment.

As shown in FIG. 13, flow diverter 401 may be disposed in compliant tubing 141 such that compliant tubing 141 is bisected into a lower portion (141a) and an upper portion (141b). Compliant tubing lower portion 141a may interface with primary flowrate control actuator 222 disposed in the control system assembly (as described elsewhere herein) to variably pinch or release compliant tubing 141a to disallow or allow drainage of the target body fluid. Compliant tubing upper portion 141b may interface with secondary flow shutoff actuator 230. Flow diverter 401 may divert flow from fluid drainage branch 402 to fluid sampling branch 403. Fluid sample tube 404 (microbore tubing, peristaltic pump tubing, etc.) may be fluidly connected at one end to fluid sampling branch 403 of flow diverter 401 and at an opposite end to fluid sample dispensing assembly 420.

Fluid sample dispensing assembly 420 may comprise frame 421, guide rod 422, lead screw 423, and dispensing nozzle 424. Lead screw 423 may interface with lead screw actuator 425 (motor, etc.) disposed in the control system assembly such that rotational motion of lead screw 423 is converted to translational motion of the dispensing nozzle 424 by any suitable technique.

Fluid sample tube 404 may be further disposed between rollers 431 and occlusion bed 432 of peristaltic pump 430 such that sample(s) of a known volume may be withdrawn from the target body fluid and dispensed onto microfluidic assay 440 by any suitable technique.

Microfluidic assay 440 may comprise one or more wells 441 for receiving fresh body fluid samples for analysis and one or more wells 442 for receiving stagnant fluid remaining in fluid sample tube 404 from a previous sampling event.

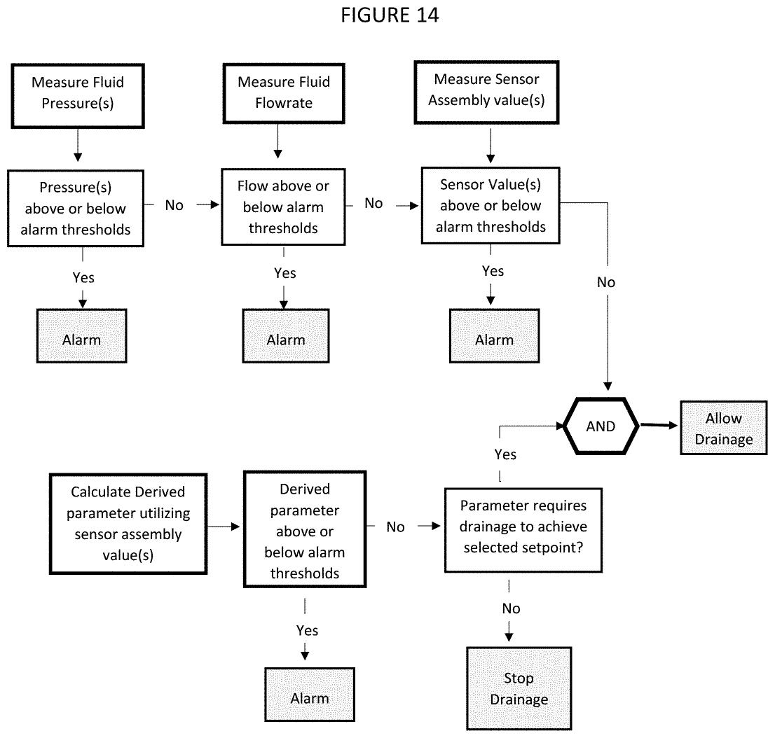

The disclosed system may comprise several operating modes depending on the type of therapy being delivered and/or the sensing modalities employed. A simplified operational diagram of a representative embodiment of the disclosed system is presented in FIG. 14.

The disclosed system may be configured to provide controlled therapeutic drainage of a body fluid according to a user-defined pressure set point while monitoring user-defined alarm threshold values for parameters such as pressure and flowrate. As depicted in FIG. 14, the system may be further configured to monitor the output of one or more sensors for the detection of one or more analytes, physiological parameters or motion, and to assert alarm(s) or notification(s) to the user when such analytes, contaminants, or toxins are detected. The system may be further configured to provide controlled therapeutic drainage or recirculation of a body fluid according to machine learning algorithm(s) or user-defined targets utilizing the detection or concentration of the analytes, contaminants, or toxins. For example, in the case of a subdural bleed or subarachnoid hemorrhage, the disclosed system may measure blood concentration in the CSF continuously or at defined intervals and drain CSF whenever the concentration exceeds an acceptably low level. The detection of the concentration of blood may be measured by an analyte sensor or by a physiological sensor optically evaluating the color of the CSF or identifying fluid composition using photo sensing at various wavelengths.

System operating mode(s) may be implemented using a combination of hardware (e.g., microcontroller, microprocessor, FPGA, SOM, etc.) and software/firmware.

The wearable motion sensing assemblies of the disclosed system may be located within the wearable pressure sensor assemblies 104 or may be a separate wearable assembly.

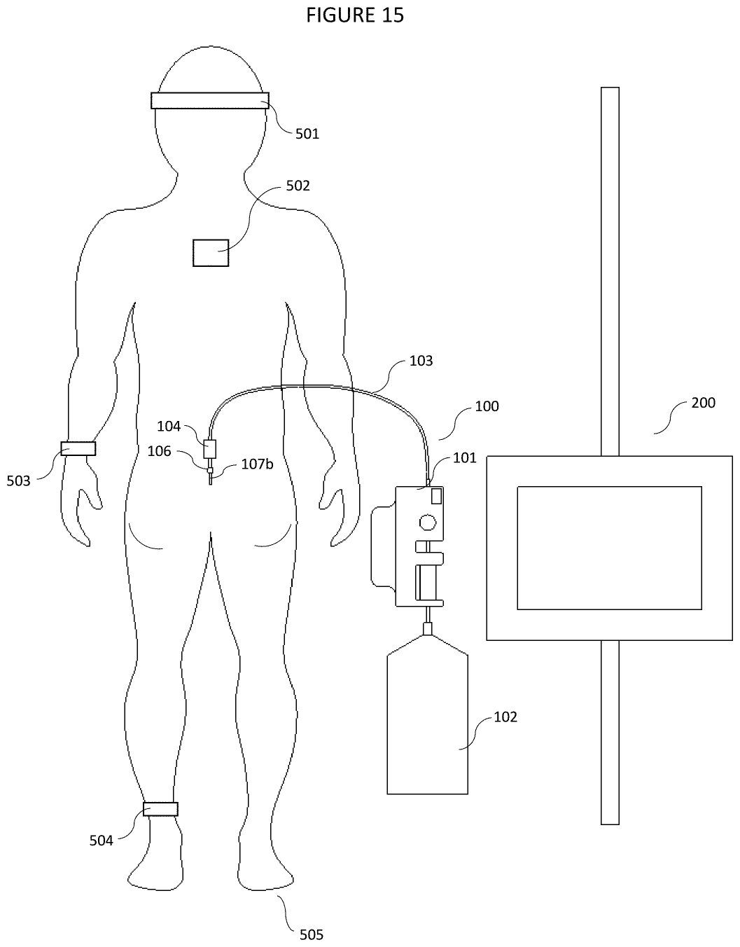

In certain embodiments, the system may comprise one or more wearable motion sensing assemblies configured for tracking movement of one or more body parts. As depicted in FIG. 15, wearable motion sensing assemblies may be configured for attachment to a patient wrist (503), ankle (504), torso/spine (502), head (501), feet (505) or any other suitable location useful for detailed tracking of patient movement for gait analysis or other related diagnostic purposes. Movement tracking assemblies may be affixed to the patient by any suitable means known in the art (adhesive patch, hook-and-loop fastener, elastic band, suture, etc.), may be configured to communicate with the control system assembly via any suitable wired (I2C, UART, etc.) or wireless (Bluetooth, Wi-Fi, infrared, etc.) connection protocol known in the art, may be powered by any suitable means known in the art (battery, wired DC power source, etc.), and may be either durable or disposable. Movement tracking assemblies may contain one or more motion sensing devices (e.g., accelerometer(s), gyroscope(s), etc.). These assemblies may also be used in combination to create a 2D, 3D or 4D model of the movement. For example, using data from the wearable assemblies 104, 501, 502, 503, 504 and 505, the control system or remote computer and user interface may calculate the ambulatory gait (stride length, stride symmetry, shuffling, etc.) and/or analyze changes in gait over time and communicate these to the user. In further embodiments, the disclosed system may combine motion data with pressure and drainage data to assess the probability of Normal Pressure Hydrocephalus (NPH) and the likelihood that treating with an implanted shunt will be successful. The disclosed system may accomplish this by evaluating gait at the start of drainage and the end of 48-hour or 72-hour drainage as is done now, improving on the art by automating evaluation and quantifying change to significantly reduce interrater variability. In further embodiments, control assembly 200 of the disclosed system may continuously evaluate data produced by wearable motion sensor assemblies in combination with body fluid volume data to detect as early as possible a change in gait characteristics that would indicate a positive diagnosis for NPH. Similarly, the disclosed system may simultaneously utilize analyte detection in addition to the above data from motion sensors and volume drainage to screen for Parkinson's disease, Alzheimer's disease or other differential diagnoses for which dementia and gait abnormalities are common symptoms.

In certain embodiments, the system may employ artificial intelligence (AI) or machine-learning (ML) algorithm(s), neural network(s), or similar technologies for geospatial calculations, data analysis, determination of diagnosis, diagnostic probability calculations, differential diagnosis, etc.

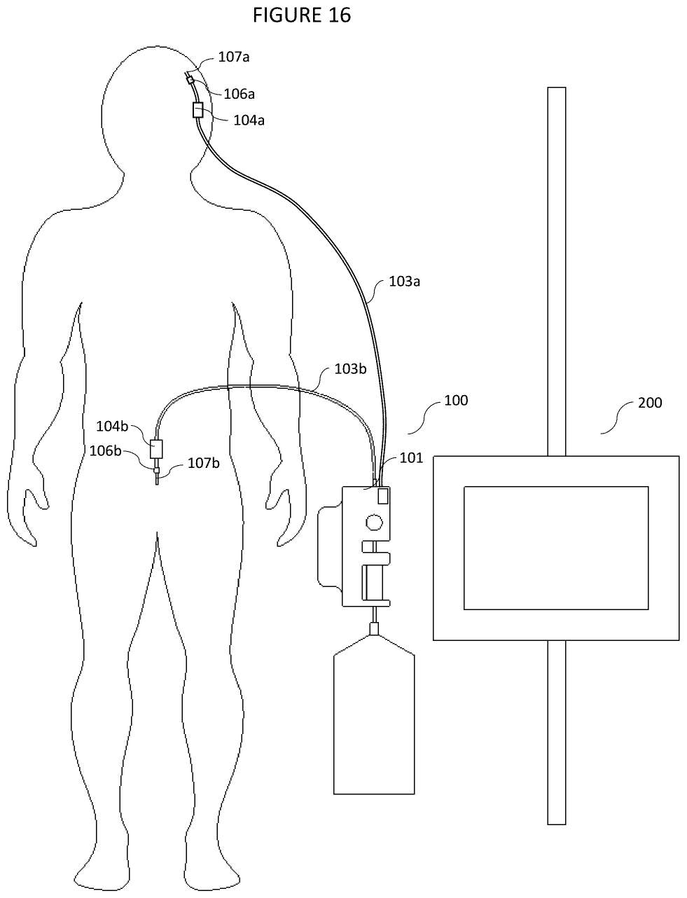

As depicted in FIG. 16, a single body fluid may be accessed at multiple anatomical locations. Ventricular catheter 107a accesses CSF in the ventricle and draining via 103a while lumbar catheter 107b accesses CSF in the intrathecal space surrounding the spinal cord and draining via 103b. This may serve to compare relative pressure or fluid characteristics to characterize circulation or lack thereof. It may also be used in combination with wearable motion sensor assemblies to characterize changes in pressure or other fluid characteristics relative to changes in patient position or movement. For example, the disclosed system may detect changes in lumbar pressure and ventricular pressure from supine to sitting to standing positions and using analytical modeling or machine learning techniques may be able to approximate supine lumbar pressure for an upright patient. Similarly, the disclosed system may detect the presence of a hole or tear in the dura causing a CSF leak by calculating the anticipated versus actual CSF pressure measured in the lumbar and ventricular regions. The disclosed system may also be configured to monitor cancerous protein levels in the same fluid in different regions of the body to narrow down the location of the likely source region. The wearable pressure sensor assemblies 104a and 104b may also be located at the thoracic and lumbar regions to allow for monitoring of blood concentration in the CSF of the thoracic region relative to the concentration in the CSF being drained in the lumbar region to determine whether appropriate clearing of blood is occurring as intended.

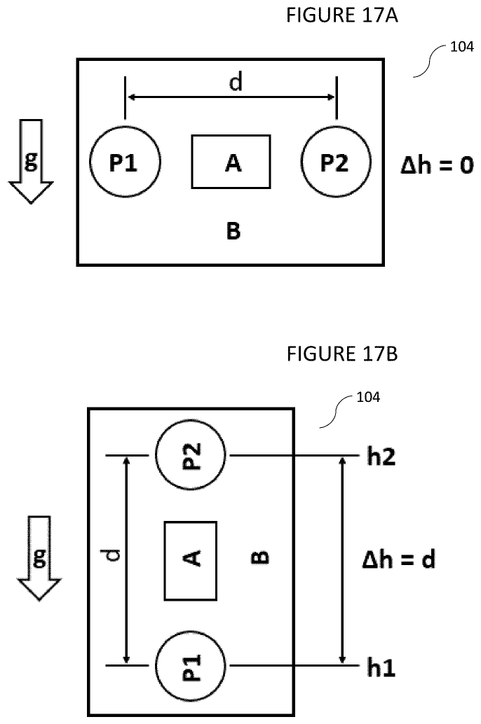

FIGS. 17A and 17B are schematic representations of wearable pressure sensor assembly 104 according to certain embodiments of the present disclosure, wherein an orientation sensor (A) and a plurality of pressure sensors (P1 and P2) are mounted at a fixed spacing distance (d) onto a rigid or semi-rigid member (B). FIG. 17A depicts wearable pressure sensor assembly 104 in a horizontal orientation with respect to gravity vector (g). FIG. 17B depicts wearable pressure sensor assembly 104 in a vertical orientation with respect to gravity vector (g).

Within certain aspects of this embodiment, the orientation sensor detects the orientation of wearable pressure sensor assembly 104 thereby facilitating calculation of an anticipated pressure differential ΔP according to the formula:

ΔPanticipated=ρ(Δh)

wherein ρ is the fluid density (e.g., the density of CSF, saline, blood, urine, etc.) and Δh is the height differential between pressure sensors P2 and P1 with respect to the gravity vector.

As depicted in FIG. 17A, when wearable pressure sensor assembly 104 is oriented horizontally with respect to the gravity vector (g), the pressure readings of the plurality of pressure sensors (P1 and P2) are substantially equivalent because the height differential (Δh) between the two pressure sensors (P1 and P2) is zero (i.e., Δh=0, so ΔPanticipated=0).

As depicted in FIG. 17B, when wearable pressure sensor assembly 104 is oriented vertically with respect to the gravity vector (g), the pressure differential between the plurality of pressure sensors (P1 and P2) is maximized because the height differential (Δh) between the two pressure sensors is also maximized (i.e., Δh=d, so ΔPanticipated=ρd).

In any wearable sensor assembly orientation other than horizontal or vertical, the height differential between the plurality of pressure sensors (P1 and P2) will vary between 0 and d based on the vertical component of orientation with respect to the gravity vector (g). The corresponding anticipated pressure differential will range from ΔPanticipated=0 to ΔPanticipated=ρd.

In certain embodiments of the control system algorithm, one or both pressure sensors may be used to determine actual measured fluid pressure, while any substantial deviation between ΔPanticipated (as described above) and ΔPactual (obtained directly via pressure sensor readings) may be used by the system to detect pressure sensor faults (electrical failure, drift in sensor accuracy, bio-fouling, etc.).

It will be apparent to one skilled in the art that the current disclosure is applicable to the measurement of gauge or absolute pressure, since either may be accomplished depending on the type of sensor used for P1 and P2, or the inclusion of separate atmospheric pressure sensor(s) outside the fluid path (such as in the control system assembly) for the calculation of gauge pressure.

The disclosed approach provides two layers of redundancy. Firstly, since each pressure sensor in wearable pressure sensor assembly 104 is located proximate to an anatomical marker for the fluid of interest, a second pressure sensor provides a direct “backup” that may allow the system to continue operating in the event that either sensor is determined to no longer be functioning normally. Secondly, the system may detect very small amounts of drift in the accuracy of the wearable sensor assembly and take appropriate action (such as notifying the user) before such errors become clinically relevant.

The disclosed approach differs from existing two-sensor systems, wherein one sensor measures the pressure in the target fluid line and a second sensor measures the pressure in a separate reference line, and wherein both pressure sensors are positioned at a location other than a relevant anatomical marker (e.g., in a pole-mounted console or hip-worn wearable). In such systems, the true pressure of the target fluid (e.g., true ICP) is calculated as the difference between the pressure in a drain line and the pressure in a separate reference line.

Two-sensor arrangements described in the prior art provide no redundancy and limited opportunities for error-checking, leaving the patient vulnerable to sensor drift and similar faults. The co-location of two pressure sensors and an orientation sensor substantially proximate to a relevant anatomical marker as described in the current disclosure provides an unprecedented level of measurement accuracy and clinical safety.

It will be appreciated that wearable pressure sensor assembly 104 must be sufficiently small and lightweight to facilitate attachment to certain anatomical markers (such as the EAM, which is located on the head, or L4-L5 located in the lumbar region of the lower back) in order to achieve practical use. As such, the use of sufficiently small pressure sensors, which are suitable for extended contact with body fluids, and which are also of sufficient accuracy and precision as to enable clinical utility, is critical to achieving the disclosed embodiments. Furthermore, the spacing distance between the sensors must be sufficiently small as to facilitate a suitable overall footprint for the assembly, which places further constraints on the precision of the pressure sensors to enable useful drift detection as described elsewhere herein. For example, a spacing distance on the order of a few centimeters is only useful if the pressure sensors are able to resolve pressure differences on the order of a few millimeters of water (mmH₂O). Such pressure sensors were unknown to the art until recently, rendering such embodiments impractical. However, due to recent technological developments, spacing distances (d) in the range of 1-2 cm are now possible, using tiny (2-3 mm wide) pressure sensors with precision on the order of ±1 mmH₂O, enabling practical embodiments of wearable pressure sensor assemblies with an overall footprint in the range of 2-5 cm² that have the characteristics described herein.

U.S. provisional patent application Nos. 63/275,232, filed Nov. 3, 2021, and 63/315,910, filed Mar. 2, 2022, to which this application claims priority, are hereby incorporated herein by reference in their entireties. The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

1. A body fluid management system, comprising:
a control system assembly for real-time monitoring of a pressure of a body fluid;
a patient interface assembly comprising at least one wearable pressure sensor assembly; and
at least one sensor assembly configured for detecting at least one parameter of the body fluid.

2. The system of claim 1 wherein the control system assembly comprises integrated control of drainage of the body fluid.

3. The system of claim 1 wherein the wearable pressure sensor assembly is configured for attaching proximate to a patient anatomical marker.

4. The system of claim 1 wherein the wearable pressure sensor assembly comprises at least one pressure sensor in direct fluid communication with the body fluid.

5. The system of claim 1 wherein the control system is configured for monitoring for changes in pressure of the body fluid.

6. The system of claim 1 wherein the at least one sensor assembly is an analyte sensor assembly in direct fluid communication with the body fluid.

7. The system of claim 6 wherein the control system assembly comprises integrated control of drainage of the body fluid.

8. The system of claim 6 wherein the analyte sensor is disposed within a sealed fluid path of the body fluid.

9. The system of claim 6 wherein the analyte sensor assembly is configured for detecting salinity of the body fluid.

10. The system of claim 6 wherein the analyte sensor assembly is configured for detecting glucose level of the body fluid.

11. The system of claim 6 wherein the analyte sensor assembly is configured for detecting protein concentration of the body fluid.

12. The system of claim 1 wherein the at least one sensor assembly is a physiological sensor assembly.

13. The system of claim 12 wherein the physiological sensor assembly is configured for detecting color of the body fluid.

14. The system of claim 12 wherein the physiological sensor assembly is configured for detecting opacity of the body fluid.

15. The system of claim 12 wherein the physiological sensor assembly is configured for detecting particulate in the body fluid.

16. The system of claim 12 wherein the physiological sensor assembly is configured for detecting oxygen concentration in the body fluid.

17. The system of claim 1 wherein the control system communicates a quantitative value derived from the sensor assembly.

18. The system of claim 1 wherein the control system communicates a qualitative parameter from the sensor assembly.

19. A body fluid management system, comprising:
a control system assembly for real-time monitoring of a pressure of a body fluid and integrated control of drainage of the body fluid;
a patient interface assembly comprising at least one wearable pressure sensor assembly; and
at least one customizable sensor assembly configured for integration of an interchangeable sensor subassembly.

20. The system of claim 19 wherein the interchangeable sensor subassembly is an interchangeable analyte detection subassembly.

21. The system of claim 19 wherein the interchangeable sensor subassembly comprises a printed circuit board.

22. The system of claim 21 wherein the interchangeable sensor subassembly communicates with the control system assembly.

23. A body fluid management system, comprising:
a control system assembly for real-time monitoring of a pressure of a body fluid and integrated control of drainage of the body fluid;
a patient interface assembly comprising at least one wearable pressure sensor assembly; and
at least one wearable motion sensor assembly.

24. The system of claim 23 wherein the patient interface assembly comprises at least one orientation sensor configured to detect an orientation of a body cavity containing the body fluid and movement of the body cavity.

25. The system of claim 23 wherein the wearable motion sensor assembly comprises at least one motion sensor configured to detect movement of the body.

26. The system of claim 23 wherein the at least one wearable motion sensor assembly comprises two wearable motion sensor assemblies configured to be worn one on each ankle.

27. The system of claim 23 wherein the at least one wearable motion sensor assembly comprises two wearable motion sensor assemblies configured to be worn one on each shoe.

28. The system of claim 23 wherein the at least one wearable motion sensor assembly is configured to be worn at the lumbar region.

29. The system of claim 23 wherein the control system assembly is configured to derive gait from patient interface assembly inputs.

30. The system of claim 29 wherein the control system assembly is configured to detect normal pressure hydrocephalus that may be improved by a shunt.
